(19) United States
(12) Patent Application Publication
(10) Pub. No.: US 2014/0142664 A1
(43) Pub. Date: May 22, 2014

(54) HIGHLY MULTIPLEXED OPTOGENETIC NEURAL STIMULATION USING INTEGRATED OPTICAL TECHNOLOGIES

(71) Applicant: California Institute of Technology, Pasadena, CA (US)

(72) Inventors: Michael L. Roukes, Pasadena, CA (US); Trevor Fowler, Pasadena, CA (US); Jessica L. Arlett, South Pasadena, CA (US)

(73) Assignee: California Institute of Technology, Pasadena, CA (US)

(21) Appl. No.: 14/086,790

(22) Filed: Nov. 21, 2013

Related U.S. Application Data
(60) Provisional application No. 61/728,908, filed on Nov. 21, 2012.

(51) Int. Cl. A61N 5/06

(52) U.S. Cl. CPC A61N 5/0622
USPC 607/88

(57) ABSTRACT

A neural probe is provided. The probe includes: a base that includes an optical demultiplexer, and one or more shanks extending from the base, with each shank including one or more waveguides. In the neural probe, the optical demultiplexer is optically connected to the one or more waveguides of each shank. A system including the neural probe and an optical multiplexer optically connected to the demultiplexer of the neural probe is also provided. Further, a method of emitting light from a neural probe is provided. The method includes receiving and demultiplexing multiplexed optical signals at a base of a neural probe, and emitting the demultiplexed optical signals from one or more shanks of the neural probe.

### STATEMENT REGARDING FEDERALLY SPONSORED RESEARCH OR DEVELOPMENT

This invention was made with Government support under Grant No. OD006924 awarded by the National Institutes of Health. The Government has certain rights in this invention.

### BACKGROUND

1. Field Of The Invention

The invention relates to a device for optical stimulation of neural tissue.

2. Related Art

Traditionally, measurements of neural activity were taken using simple pipette-based electrodes but, as the field progressed, so did the desire to measure networks of neurons, especially in vivo. Eventually the microelectromechanical systems (MEMS) field provided an alternative and reproducible path to integrated circuit fabrication technology and production of high density arrays of extracellular neural probes. Although extracellular recording does not allow for the same richness of information provided by intracellular recordings (i.e., only spiking can be effectively measured using extracellular electrodes), they are still the most prevalent method for measurement of neural networks due to their scalability. Accordingly, these probes are in common use today in research labs for chronic neural recording. Extracellular probes are able to independently record spikes from hundreds of neurons in the brain over periods of days or even months.

Due to the extracellular nature of implanted neural probes, it is impossible to effectively modulate neural activity in small, localized groups of specific cells by electrical means. Thus, the goal of the nascent field of optogenetics is to utilize optical means, taking advantage of its ability to interact with specifically modified neurons. Over the past decade there has been a great interest in stimulating neurons using light-activated ion channels. Light-activated channels have the capability of not perturbing damaging neurons, while allowing for light-activated modulation of neuron membrane potentials. Furthermore, light-activated channels can be delivered to individual neuron types, allowing for specific activation of targeted neurons. A growing class of proteins has been used to modulate neural signals; however the most effective stimulatory protein to date is channelrhodopsin-2. ChR-2 is a light stimulated cation channel which was discovered in the green algae C. reinhardtii. There are now tens of different proteins available for optogenetic excitation or inhibition of neurons, each optimized for a range of specific applications. [Yizhar, O., et al., Optogenetics in Neural Systems. Neuron, 2011. 71; Zhang, F., et al., Channelrhodopsin-2 and optical control of excitable cells. Nature Methods, 2006. 3(10).]

Since the spatial organization of neural structures begets a neural network's functionality, it becomes critical for experimentalists to be able to spatially, as well as temporally, activate neurons arbitrarily. In the current state of the art for neural stimulation and detection, an array is created from individual fiber tapers, the 6×6 grid of which are to be implanted into the brain of a rat or mouse. Each taper is coated with aluminum, which can be used to measure local spiking as well as local field potentials in the brain. Although this is the first multiplexed optical probe technology that combines multiplexed stimulation and spike detection in the same device, the major issue with this technique is a practical one as the array requires a 1:1 point of stimulation-to-fiber optic ratio. Commercially available technology utilizes a still cruder approach: an optical fiber is simply glued to a standard neural probe.

### SUMMARY

Although previous approaches may be sufficient for preliminary experimentation, it lacks any ability for spatial multiplexing and, especially, robust production for delivery to the neurophysiological research community.

In contrast to these previous approaches, embodiments of the present invention involve multiplexing many channels of light outside of the neural probe, delivering the multiplexed light through a single optical fiber, and performing compact demultiplexing locally, right at the base of the shank. By using a single optical fiber to the neural probe, which in some embodiments can control at least 32 or more independent points of stimulation, this technology becomes scalable in so much that a few optical fibers could potentially stimulate arbitrarily large regions of the brain. In addition, a system is provided for the spatiotemporal modulation of optical neural stimulation using integrated optical wavelength division demultiplexing.

In one aspect, a neural probe is provided. The probe includes: a base that includes an optical demultiplexer, and one or more shanks extending from the base, with each shank including one or more waveguides. In the neural probe, the optical demultiplexer is optically connected to the one or more waveguides of each shank.

In relation to the neural probe: a) the demultiplexer can perform wavelength division demultiplexing of optical signals; b) the demultiplexer can include an array waveguide grating; c) where the demultiplexer includes an array waveguide grating, the array waveguide grating can demultiplex a 32 channel optical signal; d) the one or more waveguides of each shank can extend along the shank's length; e) each shank can include one or more optical emitters optically connected to the one or more waveguides of the shank; f) where a shank includes one or more optical emitters, the optical emitters can include a blunt end of a waveguide terminus, a pointed end of a waveguide terminus, a waveguide facet, or an optical grating, or a combination thereof; g) the one or more shanks, the demultiplexer, or both, can be microsized structures that include nanosized optical components; or h) any combination of a)-g).

In relation to the neural probe, the following ranges can apply: the base can have the following dimensions: 1 or more millimeters to 1 or more centimeters; the shanks can have the following dimensions: 50 μm to 250 μm wide, 1 mm to 1 cm long; the demultiplexer can have the following dimensions: between 100 μm to 1 mm on a side; the waveguides can have the following dimensions: width (or height) of 100 nm to 1 μm, and length of 10 microns to 1 or more centimeters.

In a further aspect, an optical stimulation system that includes the neural probe is provided. The system includes the neural probe and an optical multiplexer optically connected to the demultiplexer of the neural probe. In the system: a) the multiplexer can perform wavelength division multiplexing of optical signals; b) where the multiplexer performs wavelength division multiplexing, the multiplexer can include a polychromator, a monochromator, a tunable laser, a supercontinuum source with acousto-optic tunable filter arrays, or any other tunable source; c) where the multiplexer is a polychromator, the polychromator can include a digital micromirror device; or d) any combination of a)-c).

In another aspect, a method of emitting light from a neural probe is provided. The method includes receiving and demultiplexing multiplexed optical signals at or on a base of a neural probe, and emitting the demultiplexed optical signals from one or more shanks of the neural probe.

In the method: a) the multiplexed optical signals can be received from an optical multiplexer optically connected to the neural probe; b) where multiplexed optical signals are received from an optical multiplexer, the multiplexer can perform wavelength division multiplexing; c) where the multiplexer performs wavelength division multiplexing, the multiplexer can include a polychromator, a monochromator, or a tunable laser, a supercontinuum source with acousto-optic tunable filter arrays, or any other tunable source; d) where the multiplexer includes a polychromator, the polychromator can include a digital micromirror device; e) the demultiplexing can be carried out by a demultiplexer that performs wavelength division demultiplexing; f) where the demultiplexer performs wavelength division demultiplexing, the demultiplexer can include an array waveguide grating; g) the one or more shanks, the demultiplexer, or both, can be microsized structures comprising nanosized optical components; or h) any combination of a)-g).

A microsized component has at least one dimension in the 0.1 μm-100 μm range. A nanosized component has at least one dimension in the 0.1 nm-100 nm range.

### BRIEF DESCRIPTION OF THE DRAWINGS

For a more complete understanding of the present invention, reference is now made to the following descriptions taken in conjunction with the accompanying drawings, in which:

FIG. 1 is a schematic drawing of an array waveguide grating.

FIGS. 2A-2L are schematic drawings of a neural probe fabrication process.

FIG. 3 is a schematic drawing of a polychromator optical system.

FIG. 4 is a schematic drawing of a digital micromirror device.

FIG. 5 is a schematic drawing of an optical stimulation system.

FIGS. 6A-6D are an optical microscope image (6A) and scanning electron microscope images (6B-6D) of fabricated array waveguide gratings.

### DETAILED DESCRIPTION

The following application is incorporated by reference herein: U.S. Provisional Patent Application No. 61/728,908, filed on Nov. 21, 2012.

Fiber optic technology has been quite popular for some time due to high data rates and low losses sustained by optical signals, and thus significant thought has gone into integrating fiber optics with silicon-based transmitters and receivers. Essentially, integrated optics is the technology enabling the manipulation of light on chips using microfabricated structures. There are direct parallels to microelectronics integrated circuit technology, where electrons are manipulated. Currently there are a multitude of well-validated methods for coupling light on and off of silicon chips [12, 13], creating optical waveguides (the optical equivalent of electrical wires) [14], realizing multiplexers and demultiplexers [15], and optical resonators [15], to name only a few amongst a variety of applications and structures. Although much of this technology is designed for use in the telecommunications band (around 1550 nm), the inventors realize that these technologies can be adapted for different wavelengths (necessary for optogenetics) by using alternative materials to silicon.

### Planar Lightwave Circuits

Planar lightwave circuits (PLCs) can be used for on-shank wavelength division demultiplexing. The basic unit of integrated optical structures is the waveguide. Similar to electrical wires, waveguides act as pipes to conduct light from one place to another. Created from non-absorbing dielectric materials, optical waveguides are most often produced by etching a thin film into long, rectangular slabs in which light can be confined using the principle of total internal reflection. This requires that the waveguide material has an index of refraction greater (ideally much greater) than that of the surrounding cladding. Air is commonly used as cladding, with an index of 1, so most optically clear dielectric materials can guide light effectively.

One of the most ubiquitous applications of PLCs in the telecommunications industry is wavelength division multiplexing (WDM). WDM is an optical technique which divides a region of the electromagnetic spectrum into a number of channels which can be used to address a single or multiple devices assigned to that spectral segment. Signals are multiplexed by assigning each signal to a channel where light in the corresponding spectral segment, modulating the amplitude of each channel, and combining all of the channels into a single optical fiber. At the terminus of the optical fiber another device is designed to break the signal into spectral components. The WDM strategy is advantageous in the current application because channelrhodopsin-2 has a broad action spectrum, thus allowing for many wavelengths (from 450 to 500 nm at over 80% efficacy) to stimulate neurons. Furthermore, most WDM strategies are passive, reducing the complexity of the optical devices on the neural probe itself. In various embodiments, WDM is used to define a number of channels, each of which correspond to a specific point of stimulation, and combine those signals into a single fiber optic which will conduct that information to a neural probe, where the signals will be demultiplexed on the probe

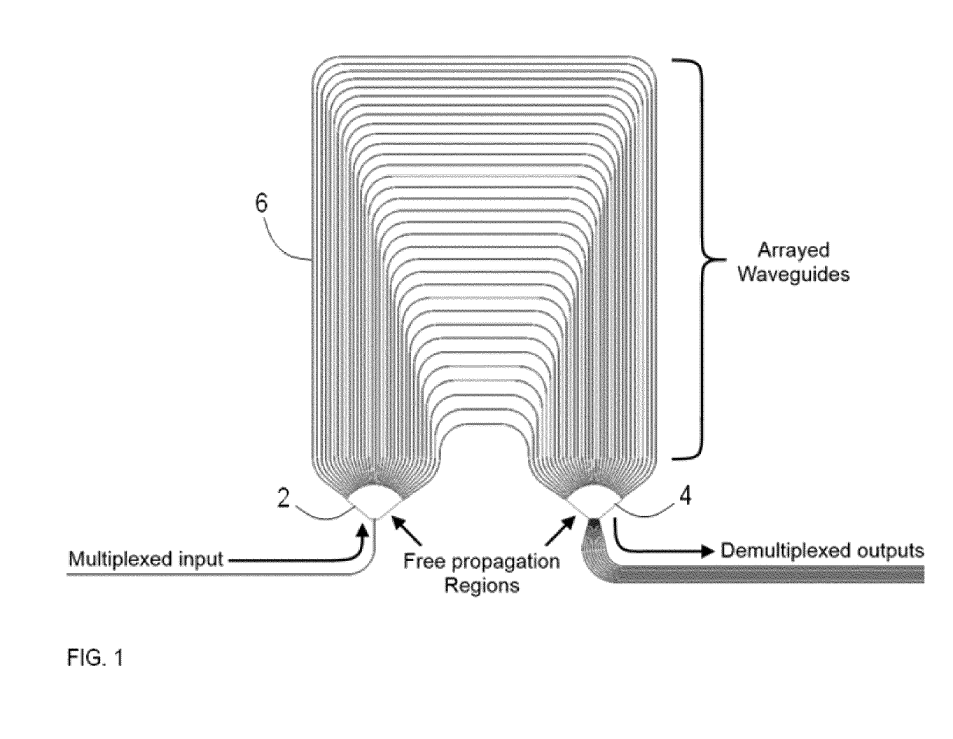

In some embodiments, on-probe demultiplexing is accomplished using the array waveguide grating (AWG). AWGs have excellent performance, are robust, are simple to fabricate and are used extensively in the telecommunications industry. AWG technology is an integrated dense wavelength division scheme which utilizes optical waveguides as delay lines to act as a grating and direct light to a number of receiver waveguides (similar conceptually to a phased array in radio communications). An example of an AWG can be seen in FIG. 1. The AWG device consists of three regions, two slab waveguides 2,4 which are used for shaping of the input beam and divergence on the receiving end, and an array 6 of waveguides, with each waveguide slightly longer than the previous. Light begins in the waveguide on the left and is emitted into the first multi-mode region of the AWG. The purpose of this region is to selectively illuminate the array of waveguides which form the grating such that they begin with the same phase. The light then propagates through a series of delays created in each subsequent waveguide, which act in a similar fashion to a diffraction grating. However unlike a grating where the path length difference is induced by illuminating the facets of a grating at an angle; instead waveguides of slightly different lengths are used to create the delays. The end result is the same as in a traditional grating, where the phase of the light emerging from the grating produces an interference-induced wavelength dependent focus on the image plane of the spectrometer. When a secondary array of waveguides is placed at the focal plane of the AWG spectrometer the device acts as a wavelength dependent demultiplexer. Thus by shaping (i.e., optically multiplexing) the spectrum off of the neural probe, individual waveguides (and thereby individual points of stimulation) can be selectively illuminated on the shank, providing spatial control of stimulation.

AWGs have been demonstrated in the telecommunication band (around 1550 nm) with hundreds of channels, and thousands when using cascaded AWG systems. AWGs were designed by the inventors using the method described in M. K. Smit: “New focusing and dispersive planar component based on an optical phased array,” Electron. Lett. 24, 385-386 (1988) to assess the feasibility of producing high density AWGs practical for neural applications at around 485 nm. Given a channel density of 1 nm, fabrication limit of 50nm minimum spacing between waveguides, 200 nm×200 nm waveguides (determined via simulation), maximum cross talk of −15 dB, and a 3 dB roll off, it was determined that AWGs are feasible up to at least 32×1 nm channels at a central wavelength of 480 nm, potentially more by using different geometries than the one chosen or by cascading multiple AWGs. It was also found that a typical AWG designed in this wavelength range has a footprint on the order of 300 μm×300 μm, which will easily fit on the base of a neural probe.

The terminus of each waveguide can have a variety of structures depending on the desired application. The simplest terminus would be a blunt end. A blunt end projects light preferentially down along the waveguide's central axis and is then diffracted outwards. The degree of diffraction depends on the size of the waveguide. Alternatively, the waveguide can be narrowed down to a point, allowing for an approximately spherical emission pattern from the tip of the waveguide. Facets can also be etched into the waveguide to direct light out away from the shank, or to the sides of the shank. Also, grating structures can be used to diffract light terminating in the waveguide in a particular direction.

Devices for optical coupling of various components include, but are not limited to, grating couplers and v-groove fiber guides (see U.S. Pat. No. 6,819,858).

### Fabrication

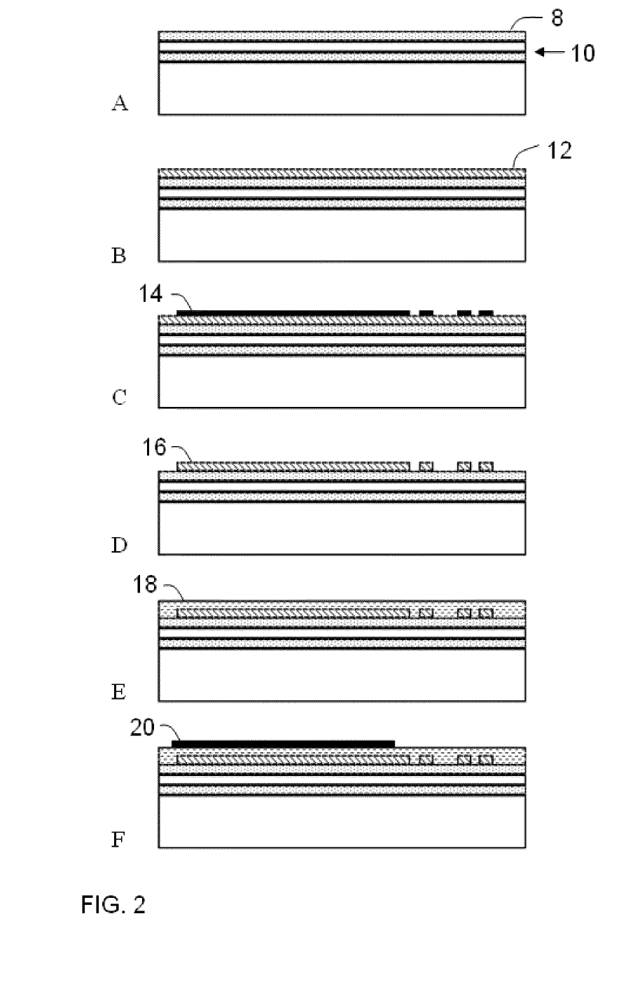

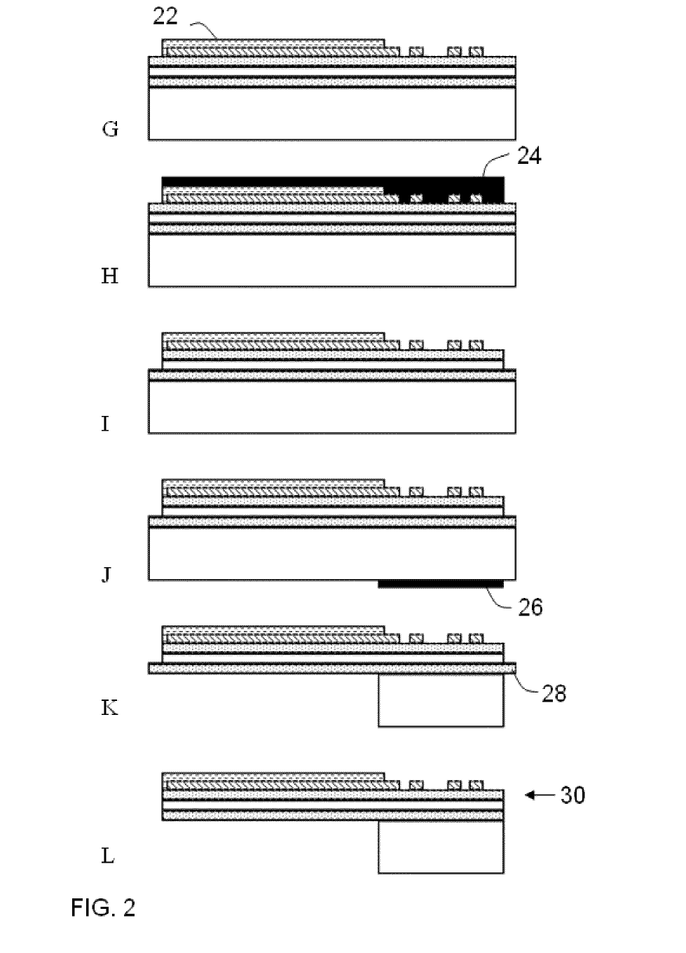

Array waveguide gratings can be fabricated using silicon nitride on top of electrophysiological neural probe shanks using micro- and nano-fabrication techniques. Probes can be fabricated on silicon-on-insulator (SOI) wafers; the thin silicon will act as the shank (the portion inserted into the brain) and the thick bottom silicon will act as the structural component for the base of the neural probe. The intermediate oxide layer acts as an etch-stop for the back-side etch which suspends the shanks The overall process is diagrammed in FIG. 2 and described below.

The first step in creating optically integrated neural probes is to thermally grow an oxide layer onto the top silicon at a thickness equal to the buried oxide. This oxide layer will act as a cladding layer for the optical waveguides and balance any residual stress created in the buried oxide. Stress balance is critical because a single oxide layer would cause the shanks to bend, increasing the risk of breaking upon insertion into the brain and making it more difficult to properly insert the neural probes into the brain.

Next, the device layer composed of silicon nitride is deposited on the thermal oxide using an LPCVD process off-site. Silicon nitride can be chosen for multiple reasons: it has favorable optical properties such as a bandgap of 4.74 eV, a bulk refractive index of 2.0, meaning that has good properties for building optical structures at blue wavelengths; and its optical properties can be controlled by a number of methods. The optical structures can be patterned by spinning on Ma-N 2401 (a negative tone e-beam resist), and subsequent exposure to an electron beam using a Leica EBPG-5000 electron beam lithography tool. The pattern can be transferred into the silicon nitride using pseudo-Bosch inductive-coupled plasma (ICP) etching, an anisotropic etch which can produce very straight sidewalls. This process produces the AWGs and waveguides on top of the oxide layer.

Next, the shanks are defined in the top silicon layer, and released from the bottom silicon substrate. First, the regions which will become the shanks are protected using a parylene-C coating. Parylene is a polymer which is superbly biocompatible, and thus will be used to coat the shanks for chronic implantation. Parylene has the added advantage of being a good choice to clad the silicon nitride waveguides (n_{paylene}=1.64, n_{SiN}=2.05). The parylene can be patterned using photolithography followed by oxygen plasma etching to expose the couplers, the AWGs and the emitters at the ends of the waveguides. Photolithography can be used to define the shape of the shanks The top silicon oxide layer can be etched using a buffered HF etch. This can be followed by a Bosch deep reactive ion etch (DRIE) to pattern the top silicon.

Then, the backside of the wafer can be patterned using photolithography to create the base of the neural probe. The pattern can be transferred using DRIE in the thick silicon layer. This will leave only the thin buried oxide, which can be removed by a short buffered-HF etch. This will also completely release the shanks from the substrate. The remaining photoresist can be stripped off, completing the fabrication process.

As shown in FIG. 2, the fabrication process can be carried out as follows: (a) thermal oxide 8 is grown on a silicon on insulator waver 10 (FIG. 2A); (b) silicon nitride 12 is deposited (FIG. 2B); (c) PMMA 14 is coated on the wafer and patterned by electron beam lithography (FIG. 2C); (d) the silicon nitride 16 is etched using a plasma RIE etch (FIG. 2D); (e) parylene 18 is coated onto the wafer (FIG. 2E); (f) photoresist 20 is applied to the wafer and is patterned (FIG. 2F); (g) parylene 22 is etched using oxygen plasma RIE (FIG. 2G); (h) photoresist 24 is applied to the wafer and is pattered to define the shanks and body of the neural probes (FIG. 2H); (i) the front side is etched with HF followed by DRIE (FIG. 2I); (j) photoresist 26 is applied to the back side of the wafer and is patterned to define the base of the neural probe (FIG. 2J); (k) the back side of the wafer is etched using DRIE (FIG. 2K); (l) the shanks are removed and dipped in HF to remove residual oxide 28, leaving the neural probe 30 (FIGS. 2K-2L).

### Spectral Shaping

To break down and encode light signals, channels can be simultaneously energized using a spectral shaping apparatus. The spectral shaping apparatus can combine diffractive optics to break down light from a broadband source into a spectrum and then spatially encode that signal using spatial light modulation. This device will herein be called a “SLM polychromator.” Through this process, an arbitrary number of frequency bands smaller than 100 pm (depending on the grating used) can be switchable at very high rates. This will allow activation of any combination of channels at rates commensurate with the switching rate of the spatial light modulator (SLM). Other technologies include monochromators, tunable lasers and mechanical polychromators, a supercontinuum source with acousto-optic tunable filter arrays, or any tunable source. However, monochromators and lasers only provide one wavelength at a time, and tuning can often be slow due to the necessity of micrometer driven mechanics. Also, mechanical polychromators have been developed in the past; however they often only support a small number of simultaneous wavelengths to be obtained from a broadband source and are often slow in tuning Since most neuroscience experiments require fast and accurate activation and deactivation of neurons, in some embodiments, the SLM polychromator is used.

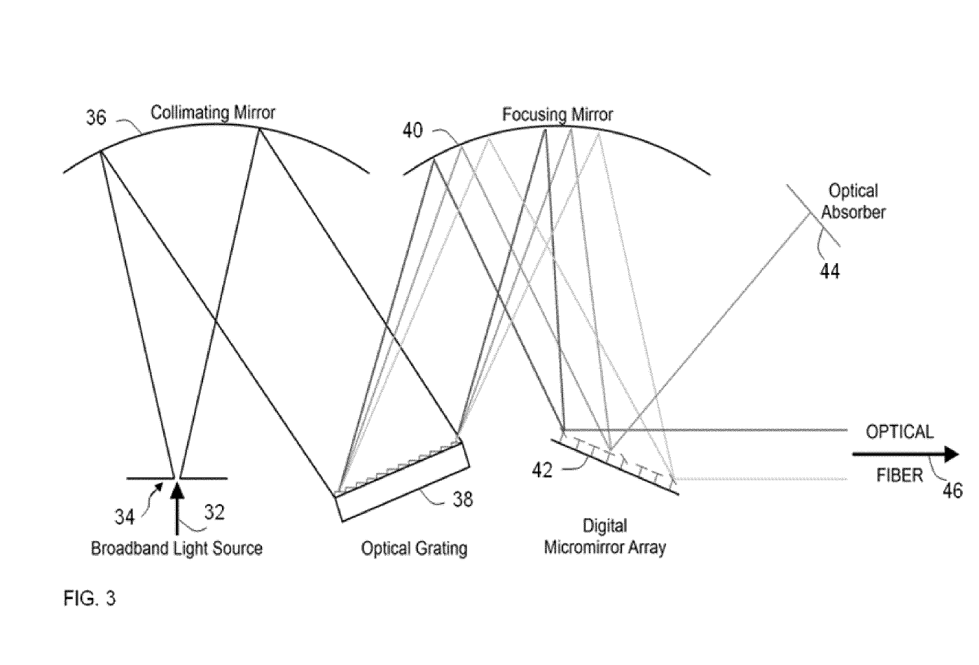

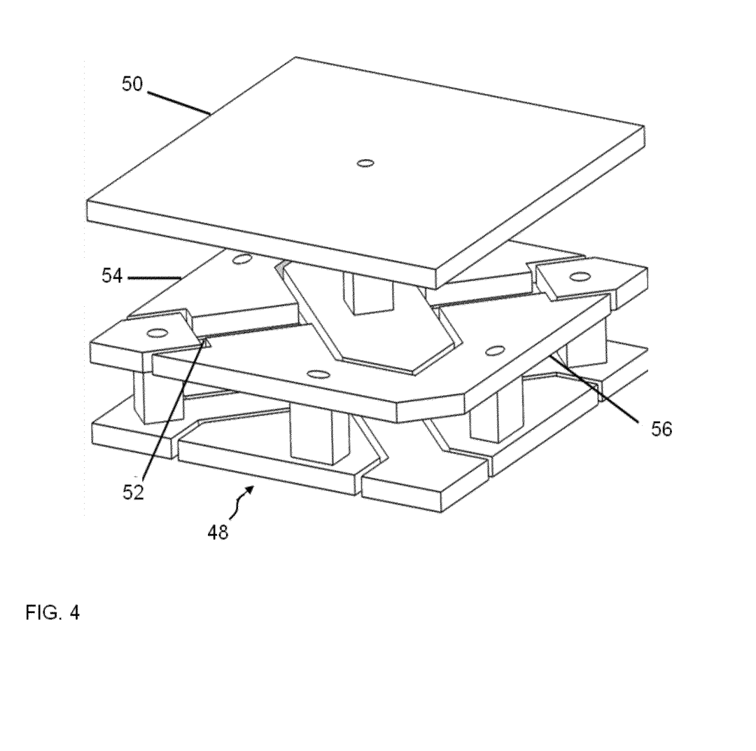

The overview of the optical setup for the SLM polychromator is shown in FIG. 3, and was originally described in Duncan, W. M., et al., The DLP™ Switched Blaze Grating: The Heart of Optical Signal Processing, SPIE Proceedings, 2003. 4983. The apparatus is in the form of a Czerny-Turner spectrograph with some modifications. Broadband light enters at a slit and is filtered to restrict incoming light to encompass no more than the excitation band for the channelrhodopsin-2. The light is collimated using a spherical mirror and projected onto a blazed (to improve diffracted light intensity) diffraction grating. The grating breaks the broadband light down into a spectrum, effectively spatially encoding the spectrum of the light. The light reflected from the grating is then captured by a camera mirror and projected onto the SLM, which allows for the spectrum to be encoded. Referring to FIG. 3, starting from the bottom left, broadband light 32 enters through a slit 34 and is collimated by a spherical mirror 36. The broadband light is projected onto a diffraction grating 38, dispersing the broadband light into a spectrum. The light is then focused by another spherical mirror 40 onto the SLM 42, which can either send light into an absorber 44 (top right) or into a fiber optic collimator 46 (bottom right).

SLMs most commonly utilize MEMS and semiconductor fabrication techniques to create a 1-d or 2-d array of elements, each of which reflect or phase-shift incoming light. In some embodiments, a digital micromirror device (DMD), which is commonly found in digital light projectors, is used. These devices (an example of which is shown in FIG. 4) utilize a large array (up to 1920×1080) of microfabricated mirrors, each of which can be rotated independently to one of two different states, +/−12°, acting effectively as an on-off switch for light. Thus, light impinging on a single pixel can be controllably reflected to two different places, for example, either into a fiber optic coupler (the on state) or a blackbody (the off state). Thus, given that the incoming spectrum is projected onto the SLM, columns of pixels can be switched on or off, allowing for spectral components (corresponding with AWG passbands) to be switch on or off likewise. Thus, the SLM polychromator enables simultaneous spectral band switching over a large spectral regions. Referring to FIG. 4, a DMD device is a rectangular array of microelectromechanical devices comprising individual pixels (an individual pixel 48 is shown in the figure). Each pixel consists of a flat mirror 50 which is attached to a doubly clamped beam 52 which acts as a torsion spring. When an electrical potential is applied to one of two electrodes 54,56 the mirror will tilt in the direction of that electrode to 11 degrees, thereby changing the incidence angle of light on the mirror. By changing the angle of incidence of light on the face of the mirror, each individual pixel can direct light in two directions, acting as an optical switch. As an array, this allows for switching of optical information.

A multiplexer can have certain specification:

i) a large spectral range allowing for at least 32 channels;

ii) operability at visible wavelengths;

iii) simultaneous and independent switching of individual channels;

iv) high optical efficiency;

v) switching times commensurate with neuronal spiking (i.e., greater than 1000 Hz).

In some embodiments, the use of a digital micromirror device can provide each of these specifications, any combination of these specifications, or all of these specifications.

In some embodiments, the overall efficiency of the system can reach a critical value of 1 mW of power delivered per channel. The efficiency of a typical high quality blazed grating is around 70%. The efficiency of the aluminum micromirrors is very flat over most of the visible regime (greater than 90% efficiency). Assuming negligible losses for other optical components, efficiencies of just below the grating efficiency (approximately 70%) are expected. A maximum power output of approximately 250 uW should be expected over a 1 nm spectral range using a xenon monochromator light source, and a blue ultra-bright LED array or super-continuum source may be used to further improve intensities, although previous work has shown that 50 uW is sufficient to stimulate local neurons. It is also noted that the spot size produced by the integrated waveguides will be much smaller than those created with an optical fiber with a core diameter of around 10 μm, which implies that using integrated waveguides a smaller dose of energy is needed to stimulate local neurons due to higher optical intensities. To stimulate larger brain regions similar to what is currently being done with optical fibers; multiple waveguides can be energized to cover a large area.

### System for Optical Stimulation

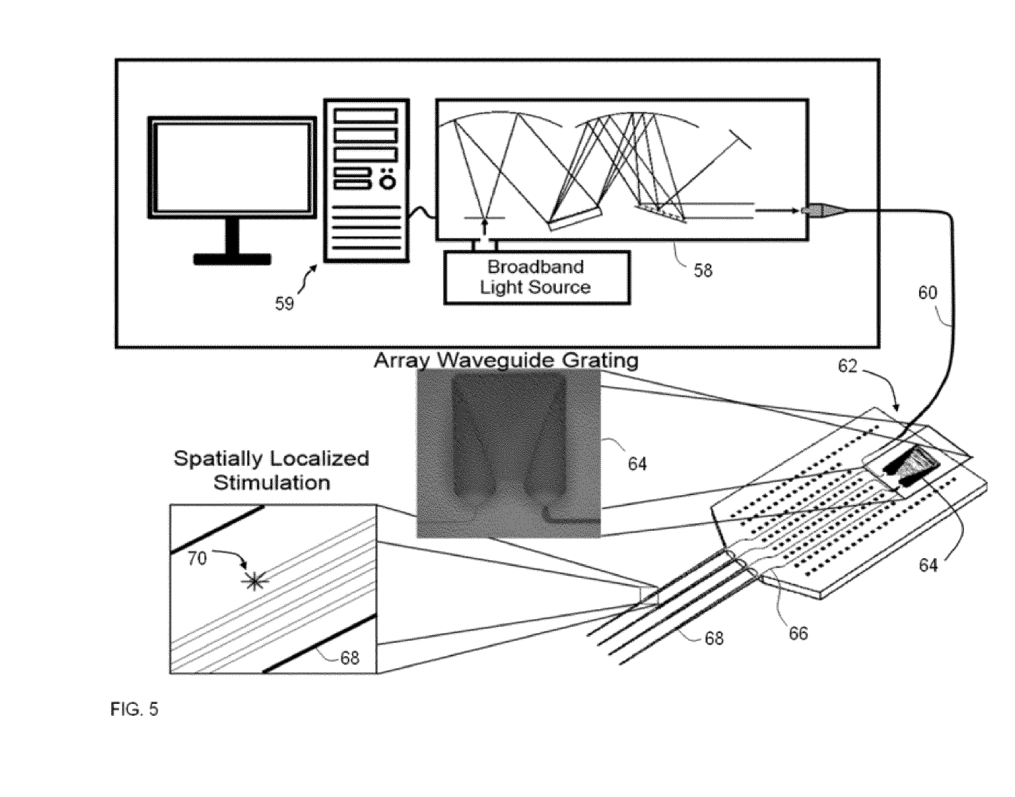

Combining array waveguide grating technology, probe microfabrication and the spectral shaping apparatus will provide a robust system for optogenetic excitation in vitro and in live animals. An embodiment of the system is shown in FIG. 5. The overall implementation of the instrument can combine the above elements into a single system allowing for arbitrary control of each individual waveguide. Light can be produced and shaped by the polychromator 58 (multiplexer) and can be collimated and injected into a fiber optic 60. A computer 59 controls the polychromator. The fiber optic can terminate at a grating coupler (for example, in some embodiments, glued using optical epoxy to a grating coupler fabricated on the base of the neural probe 62).

Simple grating couplers can be designed using MEEP (Oskooi, A. F., et al., MEEP: A flexible free-software package for electromagnetic simulations by the FDTD method, Computer Physics Communications, 2010, 181), a finite-difference time-domain solver optimized for Maxwell's equations and available on the World Wide Web at: ab-initio.mit.edu/wiki/index.php/Meep. Grating couplers utilize a periodic array of dielectric slabs to selectively diffract light from free space (or an optical fiber) into a planar waveguide fabricated on a chip. It has been shown previously that silicon nitride is an ideal material for producing very broad band gratings due to a lower refractive index than silicon, thereby reducing reflections in the coupler. Using the most basic grating coupler design, simulations show it is possible to obtain coupling efficiencies of around 20 to 25% in the visible regime with a bandwidth of over 50 nm. In some embodiments, partial etching, coatings, and other techniques can be included as other features of the design.

Referring again to FIG. 5, after light is coupled onto the neural probe 62, the on-chip array waveguide grating 64 can then demultiplex the signal and shunt the optical power to the appropriate waveguide 66, which is emitted by the shank 68 at an appropriate spot. A shank can contain multiple waveguides, and a neural probe can contain multiple shanks In some embodiments, four shanks each having eight waveguides, are provided, giving thirty-two different individually controlled points or sites of stimulation. In some embodiments, the bus waveguide can terminate at another grating coupler, which can couple into another fiber optic leading to a simple spectrometer, allowing for the resonant properties of the optical elements to be monitored.

AWGs are a mature technology in telecommunications and many of the performance issues associated with using basic AWGs have been solved, and can be modified. For example, there are multiple geometries which may be used to increase channel density and overall spectral range of AWGs (Smit, M. Progress in AWG design and technology. in Proceedings of 2005 IEEE/LEOS Workshop on Fibres and Optical Passive Components. 2005. IEEE; Okamoto, K., Fundamentals of Optical Waveguides. 2nd ed. 2006, Oxford, UK: Academic Press).

In some embodiments, non-uniformity in the pass band of each channel can be considered. If simple, straight waveguides are used, the mode conversion between the multimode region and the single mode waveguide is inefficient, producing a Gaussian pass band for the light to enter the waveguide. This can be solved through the use of parabolic receivers (Okamoto, K. and A. Sugita, Flat spectral response arrayed-waveguide grating multiplexer with parabolic waveguide horns, Electronics Letters, 1996, 32(18)). The insertion loss of AWGs can be modified by adding adiabatic tapers to the emitter and receivers to the multimode free path regions of the AWG. Optical mode invariance can be implemented by modifying the amount of over-etch of the arrayed waveguides (Okamoto, K. and A. Sugita, Flat spectral response arrayed-waveguide grating multiplexer with parabolic waveguide horns, Electronics Letters, 1996, 32(18)).

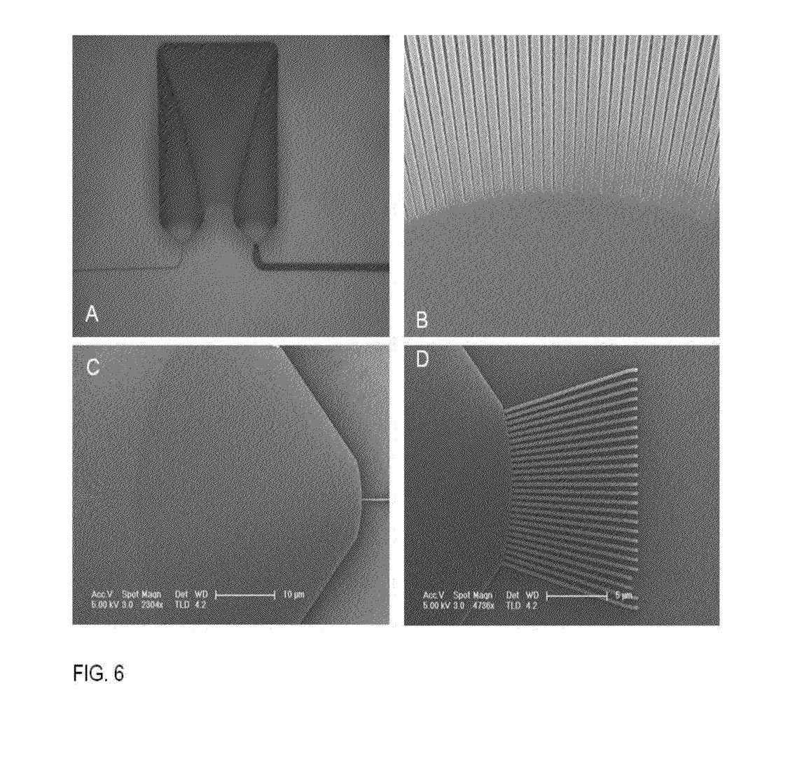

In some embodiments, pattern transfer for fabrication is considered. Lithography done on an insulating (Si₃N₄) substrate can result in local charging of the film, which can cause irregularities in the shape of the written features. This can be corrected by altering beam parameters such as write beam current or spot size. Furthermore, highly anisotropic etch recipes for silicon nitride films can be accomplished by a number of means, including pseudo-Bosch etching) or through cryogenic reactive ion etching. A pseudo-Bosch recipe has been developed for silicon nitride etching by the inventors, with good results, with a slide wall slope of approximately 87.5 degrees. The etch consists of an inductively coupled plasma utilizing a mixture of SF₆ and C₄F₈ gasses. The C₄F₈ acts to protect vertical surfaces (etched sidewalls) and SF₆ etches regions unprotected by the resist. This mixture of gasses results in a very high aspect ratio etch without the scalloping often seen by traditional Bosch type etches. The current process allows for a 50 nm line pitch at this side wall slope, as shown in FIG. 6. In some embodiments, the etch process (by tuning the gas mixture and plasma properties) can be modified to improve surface roughness further, which can improve the performance of the AWGs.

In some embodiments, the coupling of light onto the neural probes is considered. Grating couplers in the visible range have been designed and used by the inventors. Broadband, high efficiency (60% efficiency was reported) grating couplers have been developed by others using silicon nitride at 1550 nm (Maire, G., et al., High efficiency silicon nitride surface grating couplers Optics Express, 2007, 16(1); Doerr, C. R., et al., Wide Bandwidth Silicon Nitride Grating Coupler. IEEE PHOTONICS TECHNOLOGY LETTERS, 2010. 22(19)). In some embodiments, similar efficiencies at optical wavelengths through scaling principals and further optimization through iterative simulation and implementation can be obtained. Other coupling devices include v-groove fiber guides.

To characterize the AWGs and emitter beam profiles, a testing setup can be used. A fiber coupled monochromator can be used as a source, with the fiber optic terminating in a focusing collimator mounted to a multiaxis positioning system. Light from the monochromator can be focused onto the grating coupler which will couple light into a waveguide on the test chip. To measure the output of the AWGs, edge polished output waveguides can be imaged through a microscope objective and detected using a CCD detector. This will allow for the input to the AWG to be scanned (by the monochromator) and the resulting illumination pattern to be detected over multiple output waveguides.

Referring to FIG. 5, in some embodiments, emitters 70 at the tips of waveguides, especially those for implantation and stimulation of organisms in situ, can be characterized. Since the beam pattern produced at the end of the tips has effects of the number and density of activated neurons, beam patterns produced by the emitters can be investigated. Emitters can be designed and tested using imaging microscopy and beam profilometry to better understand the beam pattern and optical flux produced by the emitter tips.

In some embodiments, the polychromator can be developed using standard optical components (mirrors, diffraction grating, etc.) and a digital micromirror device. The construction of spectrometers is well known, and with the availability of high quality spectroscopic components, a SLM polychromator, in some embodiments optimized for power throughput and spectral resolution, can be produced. Measurements can be performed using a simple fiber spectrometer to optimize the output for spectral resolution and overall power throughput.

The present invention may be better understood by referring to the accompanying examples, which are intended for illustration purposes only and should not in any sense be construed as limiting the scope of the invention.

### EXAMPLE 1

Array waveguide gratings were fabricated as shown in FIG. 2.

Examples of fabricated array waveguide gratings are shown in FIG. 6. A microscope image of a 9 channel, 1 nm per channel bandwidth AWG before etching is shown in FIG. 6A, and an SEM of the arrayed waveguides is shown n FIG. 6B. Examples of a twenty-six channel, 1 nm per channel AWG input and output are shown in FIGS. 6C and 6D, respectively. Both AWGs were designed to operate at a central wavelength of 480 nm.

### REFERENCES

The following publications are incorporated by reference herein:

1. Yizhar, O., et al., Optogenetics in Neural Systems. Neuron, 2011. 71.

2. Zhang, F., et al., Channelrhodopsin-2 and optical control of excitable cells. Nature Methods, 2006. 3(10).

3. Zhang, F., et al., Red-shifted optogenetic excitation: a tool for fast neural control derived from Volvox carteri. Nat Neurosci, 2008. 11(6).

4. Boyden, E., et al., Millisecond-timescale, genetically targeted optical control of neural activity. Nat Neurosci, 2005. 8(9).

5. Sineshchekov, O. A., K.-H. Jung, and J. L. Spudich, Two rhodopsins mediate phototaxis to low- and high-intensity light in Chlamydomonas reinhardtii. P NATL ACAD SCI USA, 2002. 99(13).

6. Zhang, J., et al., Integrated device for optical stimulation and spatiotemporal electrical recording of neural activity in light-sensitized brain tissue. J Neural Eng, 2009. 6(5).

7. Zorzos, A., E. Boyden, and C. Fonstad,Multiwaveguide implantable probe for light delivery to sets of distributed brain targets. Optics Letters, 2010. 35(24).

8. Royer, S., et al., Multi-array silicon probes with integrated optical fibers: light-assisted perturbation and recording of local neural circuits in the behaving animal. Eur J Neurosci, 2010. 31(12).

9. Adar, R., et al., Optical Waveguide Comprising Bragg Grating Coupler, USPTO, Editor 1993: USA.

10. Dakss, M., et al., Grating coupler for efficient excitation of optical guided waves in thin films. Appl Phys Lett, 1970. 16(12).

11. Henry, C. H., G. E. Blonder, and R. F. Kazarinov, Glass waveguides on silicon for hybrid optical packaging. Journal of Lightwave Technology, 1989. 7(10).

12. Melloni, A., et al., Ring-resonator filters in silicon oxynitride technology for dense wavelength-division multiplexing systems. Optics Letters, 2003. 28(17).

13. Brackett, C. A., Dense wavelength division multiplexing networks: principles and applications. IEEE Journal on Selected Areas in Communications, 1990. 8(6).

14. Smit, M. Progress in AWG design and technology. in Proceedings of 2005 IEEE/LEOS Workshop on Fibres and Optical Passive Components. 2005. IEEE.

15. Okamoto, K., Fundamentals of Optical Waveguides. 2nd ed. 2006, Oxford, UK: Academic Press.

16. Takada, K., et al., 10-GHz-Spaced 1010-Channel Tandem AWG Filter Consisting of One Primary and Ten Secondary AWGs. IEEE PHOTONICS TECHNOLOGY LETTERS, 2001. 13(6).

17. Smit, M. and C. van Dam, PHASAR-Based WDM-Devices: Principles, Design and Applications. IEEE JOURNAL OF SELECTED TOPICS IN QUANTUM ELECTRONICS, 1996. 2(2).

18. Du, J., et al., High-Resolution Three-Dimensional Extracellular Recording of Neuronal Activity With Microfabricated Electrode Arrays. J Neurophysiol, 2008. 101.

19. Deshpande, S., et al., Optical properties of silicon nitride films deposited by hot filament chemical vapor deposition J Appl Phys, 1995. 77(12).

20. Haeiwa, H., T. Naganawa, and Y. Kokubun, Wide Range Center Wavelength Trimming of Vertically Coupled Microring Resonator Filter by Direct UV Irradiation to SiN Ring Core. IEEE Photonics Technology Letters, 2004. 16(1).

21. Wang, T.-J., Y.-H. Huang, and H.-L. Chen, Resonant-Wavelength Tuning of Microring Filters by Oxygen Plasma Treatment. IEEE PHOTONICS TECHNOLOGY LETTERS, 2005. 17(3).

22. Loeb, G., et al., Paryelen as a chronically stable, reproducible microelectrode insulator. IEEE Trans. Biomed. Eng., 1977. 24(2).

23. Duncan, W. M., et al., The DLP™ Switched Blaze Grating: The Heart of Optical Signal Processing. SPIE Proceedings, 2003. 4983.

24. Dudley, D., W. Duncan, and J. Slaughter, Emerging Digital Micromirror Device (DMD) Applications. SPIE Proceedings, 2003. 4985.

25. Maire, G., et al., High efficiency silicon nitride surface grating couplers Optics Express, 2007. 16(1).

26. Doerr, C. R., et al., Wide Bandwidth Silicon Nitride Grating Coupler. IEEE PHOTONICS TECHNOLOGY LETTERS, 2010. 22(19).

27. Okamoto, K. and A. Sugita,Flat spectral response arrayed-waveguide grating multiplexer with parabolic waveguide horns. Electronics Letters, 1996. 32(18).

28. Cheben, P., et al., A high-resolution silicon-on-insulator arrayed waveguide grating microspectrometer with sub-micrometer aperture waveguides. Optics Express, 2007. 15(5).

29. M. K. Smit: “New focusing and dispersive planar component based on an optical phased array,” Electron. Lett. 24, 385-386 (1988)

30. U.S. Pat. No. 6,819,858

Although the present invention has been described in connection with the preferred embodiments, it is to be understood that modifications and variations may be utilized without departing from the principles and scope of the invention, as those skilled in the art will readily understand. Accordingly, such modifications may be practiced within the scope of the invention and the following claims.

What is claimed is:

1. A neural probe, comprising
a base comprising an optical demultiplexer, and
one or more shanks extending from the base, each shank comprising one or more waveguides,
wherein the optical demultiplexer is optically connected to the one or more waveguides of each shank.

2. The neural probe of claim 1, wherein the demultiplexer performs wavelength division demultiplexing of optical signals.

3. The neural probe of claim 2, wherein the demultiplexer comprises an array waveguide grating.

4. The neural probe of claim 3, wherein the array waveguide grating demultiplexes a 32 channel optical signal.

5. The neural probe of claim 1, wherein the one or more waveguides of each shank extend along the shank's length.

6. The neural probe of claim 1, wherein each shank comprises one or more optical emitters optically connected to the one or more waveguides of the shank.

7. The neural probe of claim 6, wherein the one or more optical emitters comprise a blunt end of a waveguide terminus, a pointed end of a waveguide terminus, a waveguide facet, or an optical grating, or a combination thereof.

8. The neural probe of claim 1, wherein the one or more shanks, the demultiplexer, or both, are microsized structures comprising nanosized optical components.

9. An optical stimulation system, comprising
the neural probe of claim 1, and
an optical multiplexer optically connected to the demultiplexer of the neural probe.

10. The system of claim 9, wherein the multiplexer performs wavelength division multiplexing of optical signals.

11. The system of claim 10, wherein the multiplexer comprises a polychromator, a monochromator, a tunable laser, a supercontinuum source with acousto-optic tunable filter arrays, or another tunable source.

12. The system of claim 10, wherein the multiplexer comprises a polychromator comprising a digital micromirror device.

13. A method of emitting light from a neural probe, comprising
receiving and demultiplexing multiplexed optical signals at or on a base of a neural probe, and
emitting the demultiplexed optical signals from one or more shanks of the neural probe.

14. The method of claim 13, wherein the multiplexed optical signals are received from an optical multiplexer optically connected to the neural probe.

15. The method of claim 14, wherein the multiplexer performs wavelength division multiplexing.

16. The method of claim 15, wherein the multiplexer comprises a polychromator, a monochromator, a tunable laser, a supercontinuum source with acousto-optic tunable filter arrays, or another tunable source.

17. The method of claim 15, wherein the multiplexer comprises a polychromator comprising a digital micromirror device.

18. The method of claim 13, wherein the demultiplexing is carried out by a demultiplexer that performs wavelength division demultiplexing.

19. The method of claim 18, wherein the demultiplexer comprises an array waveguide grating.

20. The method of claim 13, wherein the one or more shanks, the demultiplexer, or both, are microsized structures comprising nanosized optical components.
